(19) United States
(12) Patent Application Publication
(10) Pub. No.: US 2015/0374548 A1
(43) Pub. Date: Dec. 31, 2015

(54) OPHTHALMIC SURGERY METHOD

(71) Applicant: CARL ZEISS MEDITEC AG, Jena (DE)

(72) Inventors: Matthias Wottke, Postbauer-Heng (DE); Bertram Meyer, Koeln (DE)

(21) Appl. No.: 14/767,806

(22) PCT Filed: Mar. 13, 2014
(86) PCT No.: PCT/EP2014/054969

§ 371 Date: Aug. 13, 2015

(30) Foreign Application Priority Data
Mar. 13, 2013 (DE) 10 2013 004 688.2

(51) Int. Cl. A61F 9/008

(52) U.S. Cl. CPC A61F 9/00827; A61F 9/00836; A61F 2009/00872; A61F 2009/00878

(57) ABSTRACT

A planning apparatus for generating control data for a treatment device, which produces at least one cut surface it the cornea by application of a laser apparatus, and to such a treatment device. The invention further relates to a method for generating control data for a treatment device, which produces at least one cut surface in the cornea by application of a laser apparatus. For this purpose, the planning apparatus determines a first access cut for the cap cut, and a second access cut for the lenticular cut, wherein the tissue is completely severed in the region of the access cuts.

### RELATED APPLICATIONS

This application is a National Phase entry of PCT Application No. PCT/EP2014/054969 filed Mar. 13, 2014, which application claims the benefit of priority to German Application No. 10 2013 004 688.2, filed Mar. 13, 2013, the entire disclosures of which are incorporated herein by reference.

### FIELD OF THE INVENTION

The invention relates to a planning apparatus for generating control data for a treatment device, which produces at least one cut surface in the cornea by application of a laser apparatus. The invention further relates to a treatment device, which has a planning apparatus of the type mentioned.

The invention further relates to a method for generating control data for a treatment device, which produces at least one cut surface in the cornea by application of a laser apparatus.
Finally, the invention also relates to a method of opthalmic surgery, wherein by means of a treatment device at least one cut surface is produced in the cornea with a laser apparatus.

### BACKGROUND

Various treatment methods with the aim of refraction correction of the human eye are known in the prior art. The aim of the surgical techniques is thereby to selectively alter the cornea in order to thus influence light refraction in the eye. To this end, several surgical techniques are used. The most common is currently so-called laser-assisted in situ keratomileusis, which is also abbreviated as LASIK. Here, a corneal lamella of the corneal surface is first detached on one side and folded aside. Detachment of this lamella can take place by means of a mechanical microkeratome, or also by means of a so-called laser keratome, such as is marketed by Intralase Corp., Irvine, USA, for example. After the lamella is detached and folded aside, the use of an excimer laser is provided during the LASIK operation, which removes the thus exposed corneal tissue under the lamella by means of ablation. After the volume under the corneal surface is vaporized in this manner, the corneal lamella is folded back to its original place.

The use of a laser keratome to lift the lamella is advantageous in comparison with a mechanical knife, as this improves geometric precision and reduces the frequency of clinically relevant complications. In particular, a lamella with a very much more constant thickness can be produced when laser radiation is used. The cut edge is also precisely formed, which reduces the risk for disordered healing by these boundary layers which also remain after the operation. A disadvantage of this method, however, is the fact that two different treatment devices must be used, firstly the laser keratome specifically to separate the lamella, and secondly the corneal tissue vaporizing laser.

These drawbacks have been eliminated in a process recently implemented by Carl Zeiss Meditec AG and designated with the abbreviation FLEx. In this method for lenticular extraction, a cut geometry is formed in the cornea by means of a short-pulse laser, preferably a femtosecond laser, which separates a corneal volume (so-called lenticule) in the cornea. This is then manually removed by the surgeon after the lenticule-covering lamella has been folded aside. The advantage of this method lies firstly in that the cut quality is again improved through use of the femtosecond laser.

Secondly, only one treatment device is necessary; the excimer laser is no longer used.

A further development of the FLEx method is referred to in the literature as the SMILE method, in which no flap is created, but rather only a small opening cut serves as an access to the lenticule harbored under the so-called cap. The separated lenticule is removed through this small opening cut, so that the biomechanical integrity of the anterior cornea is less affected than with LASIK, FLEx or PRK. Additionally, in this way fewer superficial nerve fibers are cut in the cornea, which has a demonstrably favorable effect on the restoration of the original sensitivity of the corneal surface. The symptom of dry eye, often to be treated after LASIK, is thereby reduced in severity and duration. Other complications following LASIK, primarily associated with the flap (for example, wrinkles, epithelial growths in the flap bed), occur more rarely without the flap.

When generating cut surfaces in the cornea by application of laser radiation, the optical radiation effect is typically exploited such that an optical breakthrough is generated by application of individual optical pulses whose duration may be between approximately 100 fs and 100 ns. It is also known to introduce individual pulses, the energy of which is below a threshold for optical breakthrough, into the tissue or material overlaid in such a way that a separation of material or tissue is also thereby achieved. This concept of cut production in the corneal tissue allows a wide variety of cuts.

In the cut geometry of the SMILE method according to the state of the art, it has been found that due to the small opening cuts, the two cuts bounding the lenticule (cap cut and lenticular cut) cannot clearly be identified in every case, which can therefore lead to problems during removal of the lenticule. This is carried out with a spatula-shaped instrument (also called a flap lifter), and it can occur that the doctor encounters the wrong cut surface and therefore does not correctly separate the lenticule.

### SUMMARY OF THE INVENTION

An aspect of the invention is therefore to provide a planning apparatus for generating control data, a treatment device for refraction-correcting opthalmic surgery and a method for generating control data for such a treatment device, in which an optimal execution of the access cuts to the lenticule is guaranteed.

This aspect is achieved according to the invention with a planning apparatus of the aforementioned type, which is configured to define a corneal cut surface, and configured to determine a first access cut for the cap cut and a second access cut for the lenticular cut, wherein the tissue in the area of the access cuts is completely severed.

The invention is further achieved with a treatment device which has a laser apparatus, which separates at least one cut surface in the cornea by application of laser radiation according to control data, and which has a planning apparatus according to the aforementioned type for generating control data, wherein the planning apparatus determines a first access cut for the cap cut and a second access cut for the lenticular cut, wherein the tissue is completely severed in the area of the access cuts.

Finally, the invention is also achieved with a method for generating control data according to the type mentioned above, having: generation of a control dataset for the corneal cut surface for controlling the laser apparatus, wherein the planning apparatus determines a first access cut for the cap cut and a second access cut for the lenticular cut, such that the tissue is completely severed in the area of the access cuts.

The invention is finally also achieved with a method, comprising: Generation of a control dataset for the corneal cut surface, transmission of the control data to the treatment device and generation of the cutting surfaces through control of the laser apparatus with the control dataset, wherein during generation of the control dataset a first access cut for the cap cut and a second access cut for the lenticular cut are determined such that the tissue in the area of the access cuts is completely severed.

The cap cut, i.e. the anterior cut running largely parallel to the corneal surface, is selected to be greater than the lenticular diameter. In addition, a second access cut is produced according to the invention which renders the lenticular cut accessible from the outside. This access cut can preferably be of a circularly segmented nature or strip-shaped.

It is thereby advantageous if the second access cut lies approximately at the diameter of the lenticular cut. Through the complete severing of the tissue in the area of the access cuts, finding and reaching the respective cuts is made decisively easier for the doctor, additionally the cuts are easier to identify. This complete severing of the tissue can be achieved inter alia by increasing the energy of the laser pulses, or also by a reduction in the track distance and/or spot distance of the individual laser pulses.

Furthermore, it is advantageous if the first and second access cuts are located differently with respect to the axis of the eye. According to an example embodiment, one cut is located temporally and the other cut is located inferiorly, but the combination of nasally inferior and temporally inferior is also an option.

The lenticular cut and cap cut are circular or oval and have a diameter of approximately 4 to 7 mm. The cap thickness is less than 300 μm, preferably between 100 μm and 200 μm. The removal of the lenticule causes a change of refraction, for example, between +10 dpt and −20 dpt, in another example between +5 dpt and −10 dpt. An additional or excludable cylinder correction and/or correction at an alternately high order is possible.

It is understood that the features mentioned above and those yet to be explained can be used not only in the specified combinations but also in other combinations or in isolation without departing from the scope of the present invention.

### BRIEF DESCRIPTION OF THE DRAWINGS

The invention is hereafter explained in more detail by way of example with reference to the accompanying drawings, which also disclose features essential to the invention.

FIG. 1 is a schematic representation of a treatment device with a planning apparatus for treatment in opthalmic surgical correction of refraction,

FIG. 2 is a schematic representation of the effect of the laser radiation which is used in the treatment device of FIG. 1,

FIG. 3 is a further schematic view of the treatment device of FIG. 1 with respect to the introduction of the laser radiation,

FIG. 4 is a schematic sectional view through the cornea to illustrate the removal of the corneal volume in connection with the ophthalmic surgical correction of refraction,

FIG. 5 is a schematic representation in view of the structure of the treatment unit of FIG. 1 with particular reference to the planning apparatus present there,

FIG. 6 is a schematic representation of a SMILE cut geometry according to the prior art

FIG. 7 is a schematic representation of a SMILE cut geometry according to an example embodiment of the invention.

FIG. 8 is a schematic representation of a SMILE cut geometry according to a further example embodiment of the invention.

### DETAILED DESCRIPTION

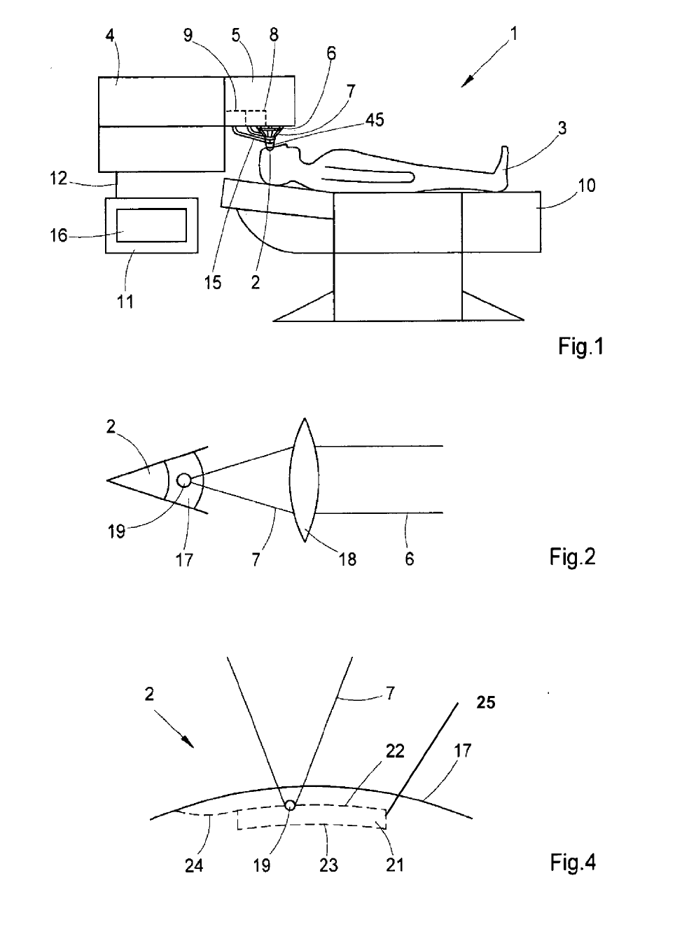

A treatment device for ophthalmic surgery is shown in FIG. 1 and provided with the general reference character 1. The treatment device 1 is designed for the introduction of laser cuts on an eye 2 of a patient 3. For this purpose, the treatment device 1 has a laser apparatus 4, which emits a laser beam 6 from a laser source 5, which is directed as a focused beam 7 in the eye 2 and the cornea. The laser beam is preferably for example a pulsed laser beam with a wavelength of between 300 nanometers and 10 micrometers. Furthermore, the pulse length of the laser beam 6 lies in the range between 1 femtosecond and 100 nanoseconds, whereby pulse repetition rates of 50 to 5000 kHz and pulse energies between 0.01 microjoules and 0.01 millijoules are possible. The treatment device 1 thus produces a cut surface in the cornea of the eye 2 through deflection of the pulsed laser radiation. For this reason, an additional scanner 8 as well as a radiation intensity modulator 9 are provided in the laser apparatus 4 and the laser source 5.

The patient 3 is located on a support 10 which is adjustable in three dimensions in order to align the eye 2 to match the incidence of the laser beam 6. In a preferred construction, the support 10 is adjustable by motor.

The control can especially be carried out by a control unit 11, which generally controls the operation of the treatment device 1, and is connected thereto via suitable data connections, for example connection lines 12, to the treatment device. Of course, this communication can also occur via other means, such as optical fibers or wirelessly. The control unit 11 performs the appropriate settings, time control of the treatment device 1, in particular the laser apparatus 4, and thus accomplishes the relevant functions of the treatment device 1.

The treatment device 1 further comprises an additional fixing apparatus 15 which positionally fixes the cornea of the eye 2 across from the laser device 4. This fixing apparatus 15 can hereby comprise a known contact lens 45, to which the cornea is applied by means of negative pressure and which imparts to the cornea a desired geometric shape. Such contact lenses are well known to those skilled in the art, for example, from DE 102005040338 A1. The disclosure of this document is, as far as the description of a construction of the possible contact lens 45 for the treatment device 1 is concerned, incorporated in full herein.

The treatment device 1 further has a camera, not shown here, which can record an image of the cornea 17 through the contact lens 45. The lighting for the camera can thereby occur both in the visible as well as in the infrared light range.

The control unit 11 of the treatment device 1 yet further has a planning apparatus 16, which will be explained in more detail later.

FIG. 2 shows schematically the operation of the incident laser beam 6. The laser beam 6 is focused and is incident as the focused laser beam 7 in the cornea 17 of the eye 2. A schematically shown lens 18 is provided for focusing. It effects a focus in the cornea 17, in which the laser radiation energy density is so high that in combination with the pulse length of the pulsed laser radiation 6, a non-linear effect on the cornea 17 occurs. For example, each pulse of the pulsed laser radiation 6 in focus 19 can produce an optical breakthrough in the cornea 17, which in turn initiates a plasma bubble indicated only schematically in FIG. 2. With the emergence of the plasma bubble, the tissue layer separation comprises a larger area than the focus 19, although the conditions for generation of the optical breakthrough are only achieved in focus 19. So that an optical breakthrough is produced by each laser pulse, the energy density, i.e. the fluence of the laser radiation, must lie above a certain pulse-length-dependent threshold value. This relationship is known to one skilled in the art, for example from DE 69500997 T2.

Alternatively, a tissue-separating effect can also be achieved by means of pulsed laser radiation while a plurality of laser radiation pulses are emitted in an area, wherein the focus spots overlap. A plurality of laser radiation pulses then act together in order to achieve a tissue-separating effect. However, the type of tissue separation which is used by the treatment device 1 is of no further relevance for the following description; it is only essential that generation of a cut surface take place in the cornea 17 of the eye 2.

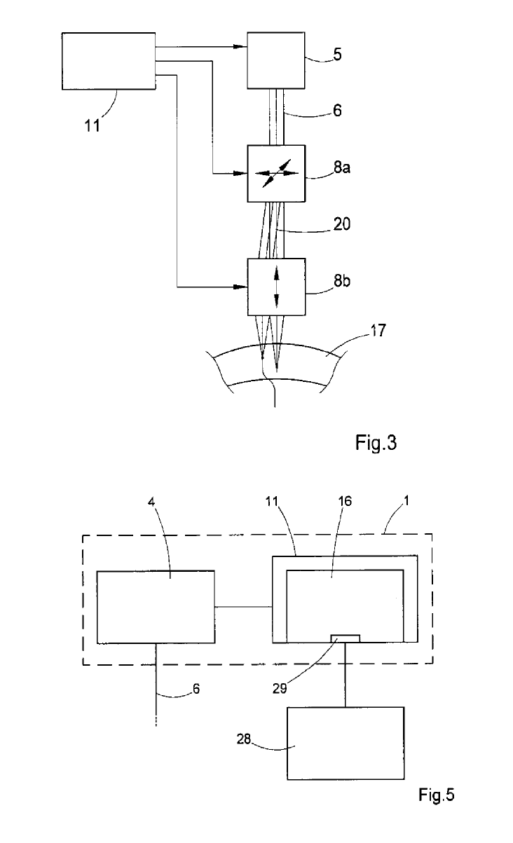

To perform an ophthalmic surgical correction of refraction, a corneal volume is removed from an area inside the cornea 17 by application of the laser radiation 6, in that the tissue layers are separated there, which isolates the corneal volume and enables the removal thereof. To isolate the corneal volume to be removed, for example, in the case of pulse-introduced laser radiation, the focus 17 of the focused laser radiation 7 is adjusted in the cornea 17. This is shown schematically in FIG. 3. The refractive properties of the cornea 17 are selectively altered by the removal of the volume, so as to achieve the correction of refraction. The volume is therefore generally lens-shaped, and is referred to as a lenticule.

In FIG. 3, the elements of the treatment device 1 are listed only to the extent necessary for understanding the generation of the cut surfaces. The laser beam 6 is, as already mentioned, brought into a focus 19 in the cornea 19, and the position of the focus 19 in the cornea is adjusted such that focusing energy from laser radiation pulses is registered in the tissue of the cornea 17 for generating cut surfaces at different locations. The laser beam 6 is preferably provided by the laser source 5 as pulsed radiation. In the method of construction of FIG. 3, the scanner is constructed in two parts and comprises an xy-scanner 8a, which in one variant is realized through two substantially orthagonally deflecting galvanometer mirrors. The scanner 8a two-dimensionally deflects the laser beam 6 coming from the laser source 5 such that a deflected laser beam 20 is present after the scanner 9. The scanner 8a thus causes an adjustment of the position of the focus 19 substantially perpendicularly to the main direction of incidence of the laser beam 6 in the cornea 17. For adjusting the depth is provided in addition to the xy-scanner 8a in the scanner 8 a further z-scanner 8b, which is designed, for example, as an adjustable telescope. The z-scanner 8b ensures that the z-position of the position of the focus 19, i.e. its position on the optical axis of incidence, is changed. The z-scanner 8b may be disposed after or before the xy-scanner 8a.

The allocation of the individual coordinates to the spacial directions is inessential for the operating principle of the treatment device 1, as is the scanner 8a diverting about mutually perpendicular axes. Rather, any scanner can be used which is able to adjust the focus 19 in a plane in which the axis of incidence of the optical radiation is not located. Furthermore, any non-Cartesian coordinate system can be used to divert or control the position of the focus 19. Examples include spherical coordinates or cylindrical coordinates.

The control of the position of the focus 19 takes place by application of the scanner 8a, 8b under control by the control unit 11, which performs appropriate adjustments to the laser source 5, the (not shown in FIG. 3) modulator 9 and the scanner 8. The control unit 11 ensures proper operation of the laser source 5 as well as the herein exemplary three-dimensional focus adjustment such that a cut surface is ultimately formed which isolates a determined corneal volume which is to be removed for correction of refraction.

The control unit 11 operates in accordance with predetermined control data, which are provided, for instance, by the laser apparatus 4, described herein only by way of example, as target points for the focus adjustment. The control data are normally summarized in a control data set. This results in geometric specifications for the cut surface to be formed, for example the coordinates of the target points as a pattern. The control data set thus also contain in this exemplary embodiment concrete locational values for the focus position adjusting mechanism, for example the scanner 8.

The production of the cut surface with the treatment device 1 is exemplarily shown in FIG. 4. A corneal volume 21 in the cornea 17 is isolated by adjusting the focus 19 in which the focused beam 7 is bundled. For this purpose, cut surfaces are formed which are exemplarily formed here as an anterior cap cut surface 22 and posterior lenticular cut surface 23. These terms are here to be understood merely as examples and are intended to establish a relationship to the conventional Lasik or Flex method for which the treatment apparatus 1, as already described, is also designed. It is only essential here that the cut surfaces 22 and 23 as well as the peripheral edge cut 25, which brings the cut surfaces 22 and 23 together at their edges, isolate the corneal volume 21. Through an opening cut 24, a corneal lamella anteriorly limiting the corneal volume 21 can further be folded such that the corneal volume 21 can be removed.

Alternatively, and essential to the present invention, the SMILE method can be used, wherein the corneal volume 21 is removed through a small opening cut, as is described in DE 10 2007 019813 A1. The disclosure of this reference is incorporated herein in full.

FIG. 5 shows the treatment device 1 schematically, and the significance of the planning apparatus 16 is to be explained in greater detail with reference thereto. In this variant, the treatment device 1 has at least two apparatuses or modules. The already described laser apparatus 4 emits the laser beam 6 onto the eye 2. The operation of the laser device 4 is carried out, as already described, fully automatically by the control unit 11, meaning that the laser apparatus 4 starts the generation and deflection of the laser beam 6 on an appropriate start signal, thereby producing cut surfaces which are constructed in the manner described. The laser apparatus 5 receives the control signal necessary for operation from the control device 11, corresponding to the aforementioned provided control data. This takes place by application of the planning apparatus 16, which is shown merely by way of example in FIG. 5 as a component of the control unit 11. Of course, the planning apparatus 16 may also be formed independently and communicate by wire or wirelessly with the control unit 11. It is only then essential that a corresponding data transmission channel between the planning apparatus 16 and the control unit 11 is provided.

The planning apparatus 16 generates a control data set, which is provided to the control unit 11 to perform the ophthalmic correction of refraction. Here, the planning apparatus uses measurement data of the cornea of the eye. This data originates in the embodiment described herein from a measuring apparatus 28, which has previously measured the eye 2 of the patient 2. Of course, the measuring device 28 may be formed in any desired manner and transmit the corresponding data to the interface 29 of the planning apparatus 16.

The planning apparatus now supports the operator of the treatment device 1 in the definition of the cut surface for isolation of the corneal volume 21. This can extend up to a fully automatic determination of the cut surfaces, which can be accomplished, for example, in that the planning apparatus 16 determines from the measured data the corneal volume 21 to be removed, defines the boundary surfaces thereof as cut surfaces and generates corresponding data therefrom for the control unit 11. At the other end of the degree of automation, the planning apparatus 16 may provide input options, where a user enters the cut surfaces in the form of geometric parameters etc. Intermediary levels provide suggestions for the cut surfaces, which the planning apparatus automatically generates and which can then be modified by an operator. In principle, all those concepts that have already been explained in the above general description section, come into use here in the planning apparatus 16.

To carry out a treatment, the planning apparatus 16 generates control data for the cut surface, which are then used in the treatment device 1.

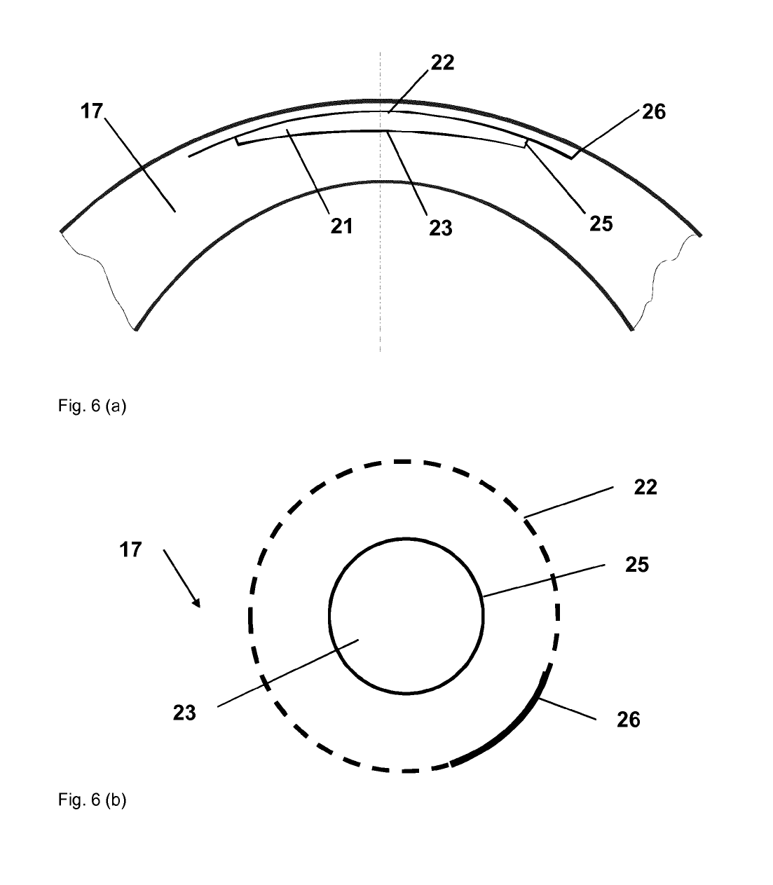

FIG. 6a shows a schematic representation of a corneal cross-section for illustrating the geometric relationships according to the prior art in the SMILE method. The cornea 17 has an anterior cap cut 22 with an opening cut 26. The posterior lenticular cut 23 isolates the lenticular volume 21, which can be removed through the opening cut 26. For this purpose, the lenticule 21 must first be completely separated in that with a spatula-shaped instrument the still-remaining tissue bridge around the cap cut 22 and the lenticular cut 23 are mechanically separated. It may hereby occur that the doctor misses the lenticular side cut forming the crossover from the cap cut 22 to the lenticular cut 23, and therefore the lenticule does not separate properly. FIG. 6b shows the cornea shown in FIG. 6a in top view.

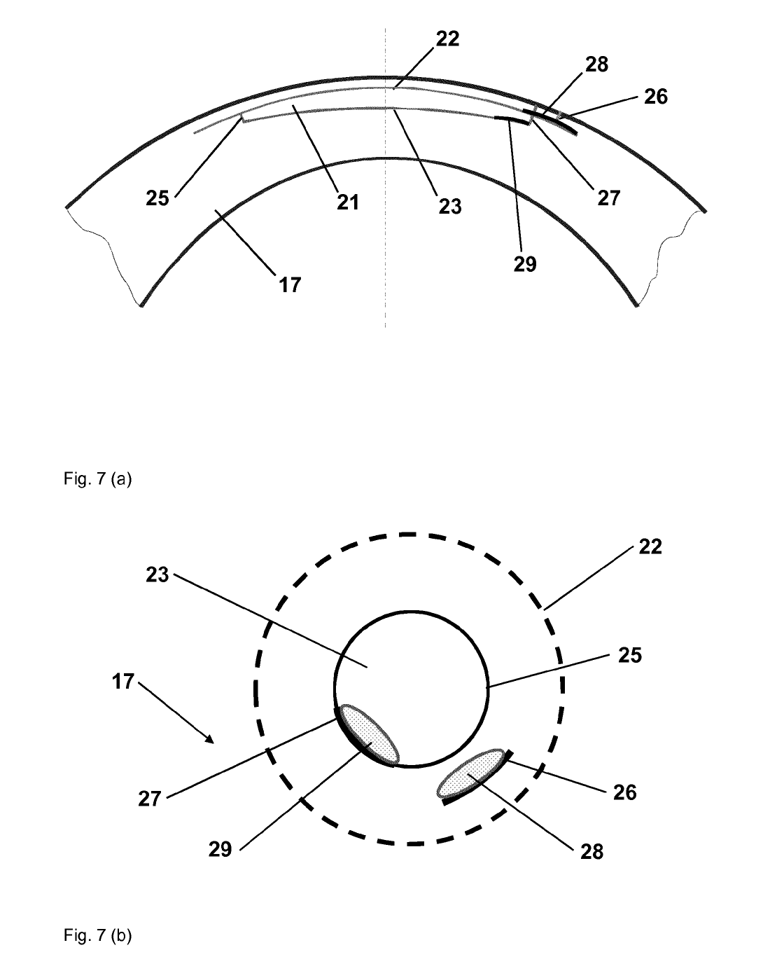

FIG. 7a shows a schematic representation of a cut geometry according to an example embodiment of the invention. Cap cut 22, lenticular cut 23 and opening cut 26 correspond to the relationships already shown in FIG. 6a. Additionally, a second opening cut 27 is provided, which allows direct access from the corneal surface to the lenticular cut 23. Additionally, the area 28 of the cap cut 22 adjacent to the opening cut 26 and the area 29 of the lenticular cut 23 adjacent to the opening cut 27 are completely severed. The risk that the doctor accidentally penetrates the cap cut 22 with the spatula-shaped instrument during separation of the lenticular cut 23 is thus largely avoided, and the complete separation of tissue permits easy and safe insertion of the instrument into the correct cut surface.

FIG. 7b shows a top view of the cornea shown in FIG. 7a. The completely severed areas 28 and 29 need not be of the form shown here, important is only a sufficient size for easy management of the spatula-shaped instrument. Additionally, (for the left eye) the usual inferior-temporal position is selected for the opening cut 26, the nasal-inferior position for the second opening cut 27, however other positions are also possible. The two cuts 26, 27 may thus also be adjacently disposed.

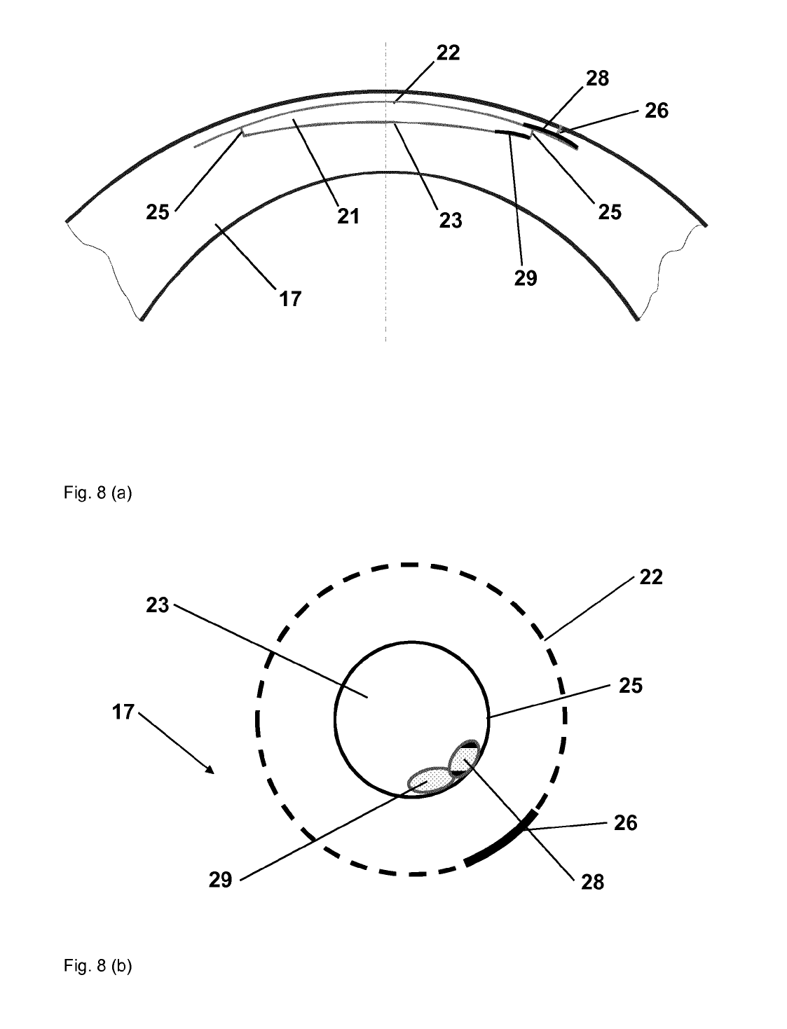

FIG. 8a shows a schematic representation of a cut geometry according to a further example embodiment of the invention. Cap cut 22, lenticular cut 23 and opening cut 26 correspond to the relationships already shown in FIG. 6a. Here, the second opening cut is directly connected to the opening cut 26 so that it is a single cut in the corneal surface. The access from the corneal surface to the lenticular cut 23 takes place via the lenticular edge cut 26. An area of the cap cut 22 adjacent to a first part of the opening cut 26 is completely severed. Further, an area of the lenticular cut 23 adjacent to a second part of the opening cut 26 is completely severed. If the doctor enters with the spatula-shaped instrument through the first part of the opening cut 26, it is guided through the completely severed area 28 into the cap cut 22. If, on the other hand, it enters through the second part of the opening cut 26, it is guided through the completely severed area 29 via the lenticular side cut 25 into the lenticular cut 23. Confusion between the two cut surfaces 22 and 23 is thus largely avoided, and the complete separation of tissue permits easy and safe insertion of the instrument into the correct cut surface. FIG. 8b shows a top view of the cornea shown in FIG. 8a. The completely severed areas 28 and 29 need not be of the form shown here, important is only a sufficient size for easy management of the spatula-shaped instrument.

In addition, it should be noted that the treatment device 1 or the planning apparatus 16 can, of course, also be concretely realized through the implementation of the method generally explained above.

A further exemplary embodiment of the planning apparatus 16 comprises a computer program or a corresponding data carrier with a computer program which realizes the planning device on a corresponding computer, such that the input of the measurement data takes place via a suitable data transmission means and the control data from this computer is transferred to the control unit 11, for which in turn data transfer means known to one skilled in the art come into question.

1.-10. (canceled)

11. A planning apparatus for generating control data for a treatment device for opthalmic surgery which generates at least two cut surfaces in the cornea by application of a laser apparatus:
wherein the planning apparatus is configured to define the corneal cut surfaces including a cap cut, a lenticular cut and an access cut, and is configured to determine a correction of refraction based on data, and is configured to generate a control data set for the corneal cut surfaces for controlling the laser apparatus;
wherein the planning apparatus is configured to determine the corneal cut surfaces such that a first access cut for the cap cut and a second access cut for the lenticular cut are determined and wherein the tissue in the area of the access cuts is completely severed.

12. The planning apparatus according to claim 11, wherein the second access cut lies approximately at a diameter of the lenticular cut.

13. The planning apparatus according to claim 12 wherein the second access cut has a position with respect to the axis of the eye deviating from the first access cut.

14. A treatment device for ophthalmic surgery, comprising:
a laser apparatus, which generates at least two cut surfaces in the cornea according to control data by application of laser radiation, and
a planning apparatus for generating the control data;
wherein the planning apparatus is configured to define the cortical cut surfaces including a cap cut, a lenticular cut and an access cut, and is configured to determine a correction of refraction based on data, and is configured to generate a control data set for the corneal cut surfaces for controlling the laser apparatus;
wherein the planning apparatus is configured to determine the cortical cut surfaces such that a first access cut for the cap cut and a second access cut for the lenticular cut are determined, wherein the tissue in the area of the access cuts is completely severed.

15. The treatment device according to claim 14, wherein the second access cut lies approximately at a diameter of the lenticular cut.

16. The treatment device according to claim 14, wherein the second access cut has a position with respect to the axis of the eye deviating from the first access cut.

17. A method of generating control data for a treatment device for ophthalmic surgery, which generates at least two cut corneal surfaces in the cornea, by application of a laser apparatus, the method comprising:
generating cornea data for correction of refraction based on the data;
defining the cut corneal surfaces; and
generating a control data set for the coronal cut surfaces for controlling a laser apparatus, wherein the cut corneal surfaces are determined, such that a first access cut for the cap cut and is second access cut for the lenticular cut are determined and tissue in the area of the first access cot and the second access cut is completely severed.

18. The method according to claim 17, further comprising generating the control data set such that the second access cut lies approximately at the diameter of the lenticular cut.

19. The method according to claim 17, further comprising generating the control data set such that the second access cut has a position with respect to the axis of the eye deviating from the first access cut.

20. A method for ophthalmic surgery, wherein, by application of a treatment device, at least two coronal cut surfaces are generated in the cornea by application of a laser apparatus, the method comprising:
generating corneal data for correction of refraction based on data;
defining the conical cut surfaces; and
generating a control data set for the cortical cut surfaces for controlling a laser apparatus, wherein the corneal cut surfaces are determined such that a first access cut for the cap cut and a second access cut for the lenticular cut are determined and tissue in an area of the first access cut and the second access cut is completely severed.

21. The method according to claim 20, further comprising generating the control data set such that the second access cut lies approximately at a diameter of the lenticular cut.

22. The method according to claim 21, further comprising generating the control data set such that the second access cut has a position with respect to the axis of the eye deviating from the first access cut.

23. A non-transitory computer readable data storage medium that is not a carrier wave or signal, comprising instructions for photodisruptive laser treatment of a cornea, the instructions being adapted to control a laser device emitting laser radiation for surgically correcting defective vision of an eye which generates at least two cut corneal surfaces in the cornea by application of a laser apparatus, the instructions comprising:
generating corneal data for correction of refraction based on data;
defining the cut corneal surfaces; and
generating a control data set for the corneal cut surfaces for controlling a laser apparatus, wherein the cut corneal surfaces are determined such that a first access cut for the cap cut and a second access cut for the lenticular cut are determined and tissue in the area of the first access cut and the second access cut is completely severed.

24. The non-transitory computer readable data storage. medium according to claim 23, the instructions further comprising generating the control data set such that the second access cut lies approximately at the diameter of the lenticular cut.

25. The non-transitory computer readable data storage medium according to claim 24, the instructions further comprising, generating the control data set such that the second access cut has a position with respect to the axis of the eye deviating from the first access cut.

26. A non-transitory computer readable data storage medium that is not a carrier wave or signal, comprising instructions for photodisruptive laser treatment of a cornea, the instructions being adapted to control a laser device emitting laser radiation for surgically correcting defective vision of an eye which generates at least two cut corneal surfaces in the cornea by application of a laser apparatus, the instructions comprising:
generating corneal data for correction of refraction based on data;
defining the corneal cut surfaces; and
generating a control data set for the corneal cut surfaces for controlling a laser apparatus, wherein the corneal cut surfaces are determined such that a first access cut for the cap cut and a second access cut for the lenticular cut are determined and tissue in an area of the first access cut and the second access cut is completely severed.

27. The non-transitory computer readable data storage medium according to claim 26, the instructions further comprising, generating the control data set such that the second access cut lies approximately at a diameter of the lenticular cut.

28. The non-transitory computer readable data storage medium according to claim 27, the instructions further comprising, generating the control data sot such that the second access cut has a position with respect to the axis of the eye deviating from the first access cut.
